(19) United States
(12) Patent Application Publication
(10) Pub. No.: US 2023/0270322 A1
(43) Pub. Date: Aug. 31, 2023

(54) ENDOSCOPE

(71) Applicant: STERILEWAVE, LLC, Port Washington, NY (US)

(72) Inventor: John Bala, Hollywood, SC (US)

(21) Appl. No.: 17/987,735

(22) Filed: Nov. 15, 2022

Related U.S. Application Data
(60) Provisional application No. 63/360,938, filed on Nov. 15, 2021.

(51) Int. Cl. A61B 1/00; A61B 1/05

(52) U.S. Cl. CPC A61B 1/00195; A61B 1/00128; A61B 1/00165; A61B 1/05

(57) ABSTRACT

Exemplary embodiments of a disposable endoscope are provided. For example, an endoscope is provided, including a tube having a proximal end and a distal end, a lumen having a proximal end and a distal end, the lumen surrounding an outer circumference of the tube, and a series of achromatic double lenses and singlet lenses within the tube from the distal end to the proximal end of the tube for conveying an image from the distal end of the tube to the proximal end of the tube. A housing is provided at the proximal end of the tube to provide a light path from the proximal end of the lumen to the distal end of the lumen from a polymer fiber optic located within the housing. A camera interface can be provided at an opposite end of the housing to receive the image from the distal end of the tube.

### CROSS-REFERENCE TO RELATED APPLICATIONS

This application relates to and claims priority from U.S. Provisional Patent Application Ser. No. 63/360,938 filed Nov. 15, 2021, the entire disclosure of which is hereby incorporated herein by reference.

### FIELD OF THE DISCLOSURE

The present disclosure relates to exemplary embodiments of an endoscope, and more particularly, to exemplary embodiments of a disposable endoscope.

### BACKGROUND INFORMATION

Endoscopes are devices used in the field of medicine that allow for, e.g., visual examination inside of the body cavity for examining organs and tissue, for diagnostic sampling of tissue, for safe manipulation of instruments, and for performing minimally invasive surgery. Endoscopes are widely used in abdominal, urological and general surgical procedures. The elements of an endoscope typically include an optical telescope for viewing the image from inside of the body cavity and a means for illuminating the area viewed by the telescope. The telescope designs generally use a combination of lenses and glass rods to transmit the image from the body cavity to a viewing eyepiece. In recent years, use of the eyepiece has been replaced with electronic cameras with the images displayed in real time on a video monitor.

Illumination of the body is typically performed by a fiber optic bundle comprised of thousands of small diameter optical fibers formed into a bundle that surround the telescope. An external light source provides illumination through a fiber optic light transmitting cable that extends the length of the telescope and connects to a ferrule at the proximal end of the endoscope. Typically, the telescopes use 20-24 lenses and glass rods to relay an image from within the body cavity to a viewing eyepiece located outside the body. Each lens element requires complex anti reflection coating to reduce surface reflection losses. The anti-reflection coatings on endoscopes with large numbers of lenses and rods require multi-layer evaporated coatings consisting of 10 to 15 layers or more of different materials to achieve transmission levels of 99 plus percent in order to achieve a brightness level that can be seen clearly by the human eye through an eyepiece. Coating failures caused by autoclaving is a major cause of endoscope failures after several uses mainly caused by adhesion and moisture problems.

The complexity of a disposable endoscope having multi-layer anti reflective coatings have made either the cost or performance unattractive in the marketplace. Substitutions of plastic lenses in place of glass lenses coupled with complex multi-layer anti-reflective coatings for multiple lenses and rods, with associative spacers together with a fiber optic assembly, have made attempts at a marketable disposable endoscope unsuccessful. Therefore, there is a need for a disposable endoscope with materials and lenses that are cost-efficient and can provide for a clear image with a strong anti-reflective coating applied to the lenses.

### SUMMARY OF EXEMPLARY EMBODIMENTS OF THE DISCLOSURE

Exemplary embodiments of the present disclosure can provide for a disposable endoscope. In some exemplary embodiments, an endoscope can be provided, comprising a tube having a proximal end and a distal end, a lumen having a proximal end and a distal end, the lumen surrounding an outer circumference of the tube, a first achromatic double lens provided at the distal end of the tube, a second achromatic double lens provided within the tube and a first singlet lens provided between the first achromatic double lens and the second achromatic double lens, a third achromatic double lens provided within the tube and a second singlet lens provided between the second achromatic double lens and the third achromatic double lens, a fourth achromatic double lens provided within the tube and a third singlet lens provided between the third achromatic double lens and the fourth achromatic double lens, a fifth achromatic double lens provided within the tube and a fourth singlet lens provided between the fourth achromatic double lens and the fifth achromatic double lens, a sixth achromatic double lens provided within the tube and adjacent the fifth achromatic double lens, and a seventh achromatic double lens provided within the tube at the proximal end of the tube and adjacent the sixth achromatic double lens. In some exemplary embodiments, the lumen and tube are constructed of a rigid material. In some exemplary embodiments, each of the achromatic double lenses and singlet lenses are made of high and low refractive index glasses having a magnesium fluoride coating. In some exemplary embodiments, the endoscope further comprises a field stop provided within the tube between the first singlet lens and the second achromatic double lens.

In some exemplary embodiments, the endoscope further comprises a first spacer provided between the first achromatic double lens and the first singlet lens, a second spacer provided between the first singlet lens and the field stop, and a third spacer provided between the field stop and the second achromatic double lens. In some exemplary embodiments, the endoscope further comprises a fourth spacer provided between the second achromatic double lens and the second singlet lens, and a fifth spacer provided between the second singlet lens and the third achromatic double lens. In some exemplary embodiments, the endoscope further comprises a sixth spacer provided between the third achromatic double lens and the third singlet lens, and a seventh spacer provided between the third singlet lens and the fourth achromatic double lens. In some exemplary embodiments, the endoscope further comprises an eighth spacer provided between the fourth achromatic double lens and the fourth singlet lens, and a ninth spacer provided between the fourth singlet lens and the fifth achromatic double lens. In some exemplary embodiments, the endoscope further comprises a tenth spacer provided between the fifth achromatic double lens and the sixth achromatic double lens, and an eleventh spacer provided between the sixth achromatic double lens and the seventh achromatic double lens.

In some exemplary embodiments, the endoscope further comprises a prism provided at the distal end of the lumen configured to provide a thirty degree viewing angle. In some exemplary embodiments, the endoscope further comprises a housing provided at the proximal end of the tube, the housing having a proximal end and a distal end, and a cemented flange at the proximal end of the tube, the cemented flange connecting the lumen to the proximal end of the housing, wherein the tube extends within the housing.

In some exemplary embodiments, the endoscope further comprises an illumination ferrule provided on the housing for providing a light pathway from the proximal end of the lumen to the distal end of the lumen. The lumen is constructed of a high index, high transmission clear polymer to transfer light from the proximal end of the housing to the distal end of the lumen. The housing comprises a two-piece assembly incorporating a clam shell construction having male and female inserts that lock the two pieces to each other, wherein the tube passes through the two-piece assembly.

In some exemplary embodiments, the endoscope further comprises a transmissive polymer fiber optic provided in the housing around the tube, wherein illumination is provided from the illumination ferrule through the transmissive polymer fiber optic to transmit light from a proximal end of the lumen to the distal end of the lumen. The polymer fiber optic comprises two high index polymer elements that provides a light ring around the tube. In some exemplary embodiments, the housing comprises a front retainer at a proximal end of the housing, and a rear retainer at a distal end of the housing, wherein the front and rear retainers hold the two high index polymer elements together.

In some exemplary embodiments, the endoscope further comprises a camera adapter provided at a distal end of the housing, wherein the tube extends within the camera adapter, an eyepiece assembly connected to the camera adapter, and a camera interface provided within the eyepiece assembly and secured to the eyepiece assembly. In some exemplary embodiments, the endoscope further comprises a first lens provided within the eyepiece assembly adjacent the camera adapter, a spacer provided adjacent the first lens, and a second lens provided adjacent the spacer, wherein an image is provided from the seventh achromatic double lens through the first lens of the eyepiece assembly to the second lens of the eyepiece assembly. In some exemplary embodiments, the endoscope further comprises threads within the camera interface for securing a camera to the camera interface to receive the image from the second lens of the eyepiece assembly.

### BRIEF DESCRIPTION OF THE DRAWINGS

The foregoing and other objects of the present disclosure will be apparent upon consideration of the following detailed description, taken in conjunction with the accompanying drawings, and claims, in which like reference characters refer to like parts throughout, and in which:

FIG. 1 illustrates an endoscope according to an exemplary embodiment of the present disclosure;

FIG. 2 illustrates an internal view of a tube of the endoscope according to an exemplary embodiment of the present disclosure;

FIGS. 3(a)-3(d) illustrate a housing of the endoscope according to an exemplary embodiment of the present disclosure;

FIG. 4 illustrates an internal design of the housing of the endoscope according to an exemplary embodiment of the present disclosure;

FIG. 5(a) illustrates a zero degree viewing angle embodiment of the endoscope according to an exemplary embodiment of the present disclosure;

FIG. 5(b) illustrates a thirty degree viewing angle embodiment of the endoscope according to an exemplary embodiment of the present disclosure;

FIGS. 6(a)-6(c) illustrate an eyepiece and camera interface to be connected to the housing of the endoscope according to an exemplary embodiment of the present disclosure; and

FIG. 7 illustrates an eyepiece lens assembly according to an exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF DISCLOSURE

The exemplary embodiments of the present disclosure can provide for a disposable endoscope. The disposable endoscope can use a combination of double achromatic lenses and singlet lenses having a magnesium fluoride coating, with an illumination ferrule provided at one end. Exemplary embodiments of the various methods and apparatuses will now be described with reference to the figures. The following description of the various embodiments is merely exemplary in nature and is in no way intended to limit the scope of the disclosure, its application, or uses.

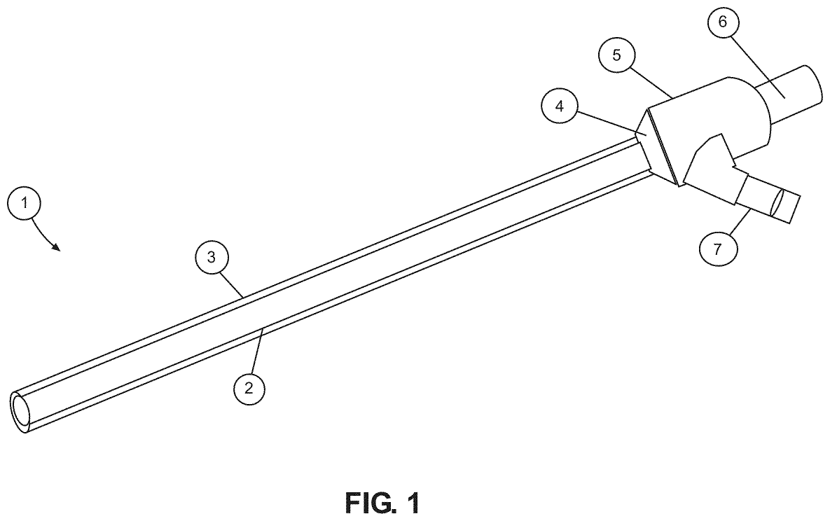

FIG. 1 illustrates an endoscope according to an exemplary embodiment of the present disclosure. As shown in FIG. 1, a disposable endoscope 1 can include a rigid telescope tube 2 surrounded by a rigid illumination lumen 3. The telescope tube 2 and illumination lumen 3 are connected to a housing 5 at a proximal end. A cemented flange 4 is connected to the end of the illumination lumen 3. The telescope tube 2 extends within the housing 5. The illumination lumen 3 can be constructed of a high index, high transmission clear polymer used to transfer light from the proximal end of the housing 5 to the distal end of the endoscope 1. An illumination ferrule 7 is connected to the housing 5 and provides a light pathway to the illumination lumen 3 from a remote light source through the ferrule 7. The telescope tube 2 and illumination lumen 3 are made from a high index, high transmission polymer material that transmits light from the ferrule 7 to the distal end of the illumination lumen 3. The illumination lumen 3 can have an approximately 5 mm diameter.

In some exemplary embodiments, an apparatus for viewing an endoscope image either by the human eye or an electronic camera through a camera adapter 6 can be provided. The apparatus can be provided on the camera adapter 6 on the proximal end of the telescope tube 2 that is integrated into the housing 5. When connecting an electronic camera to the camera adapter 6, no separate auxiliary optical device is required.

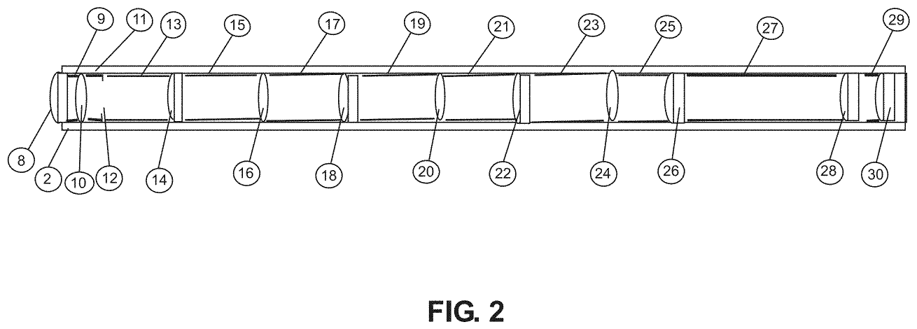

FIG. 2 illustrates an internal view of a tube of the endoscope according to an exemplary embodiment of the present disclosure. The tube 2 can be approximately 250 to approximately 400 mm in length, and in some exemplary embodiments can be 312 mm in length. An objective lens 8, which can be an achromatic doublet lens, can be provided at a distal end of the tube. A singlet lens 10 can be provided adjacent the objective lens 8, and a spacer 9 can be provided between the objective lens 8 and singlet lens 10 to precisely locate the distance between the objective lens 8 and singlet lens 10. The objective lens 8 and singlet lens 10 can provide for an objective lens system. A spacer 11 can be provided adjacent the singlet lens 10 and a field stop 12 can be provided adjacent the spacer 11. In some exemplary embodiments, the field stop 12 can be located 4.18 mm from the singlet lens 10. A spacer 13 can be provided adjacent the field stop 12.

Lens 14 is an achromatic doublet lens and can be spaced approximately 30.95 mm from the field stop 12 through spacer 13, and can be spaced approximately 28 mm to singlet lens 16 through spacer 15. Lens 18 is an achromatic doublet lens and can be spaced approximately 30 mm from singlet lens 16 through spacer 17, and can be spaced approximately 28 mm to singlet lens 20 through spacer 19. Lens 22 is an achromatic doublet lens and can be spaced approximately 30 mm from singlet lens 20 through spacer 21, and can be spaced approximately 28 mm to singlet lens 24 through spacer 23. The singlet lens 24 is a relay lens that relays an image from the distal end of the tube 2 to an achromatic doublet lens 26. The spacer 25 can provide approximately 18 mm between the singlet lens 24 and achromatic doublet lens 26.

The achromatic doublet lens 26 relays the image to achromatic doublet lens 28. A spacer 27 can provide approximately 48 mm between the achromatic doublet lens 26 and achromatic doublet lens 28. The achromatic doublet lens 28 relays the image to achromatic doublet lens 30. A spacer 29 can provide approximately 5 mm between the achromatic doublet lens 28 and achromatic doublet lens 30. The achromatic doublet lens 30 forms a focal image 4 mm to the proximal end of the tube from the end of achromatic doublet lens 30.

In some exemplary embodiments, an image that is formed can be focused directly onto an active photo cathode of an electronic camera or can be focused for human viewing with an eyepiece. The eyepiece can have additional lenses as needed. In some exemplary embodiments, all the lenses within the tube 2 are made of high and low refractive index glasses and can be anti-reflection coated with magnesium fluoride. Other anti-reflection coatings with equal or greater performance can also be used. In some exemplary embodiments, one or more of the lenses can be made of optical polymers.

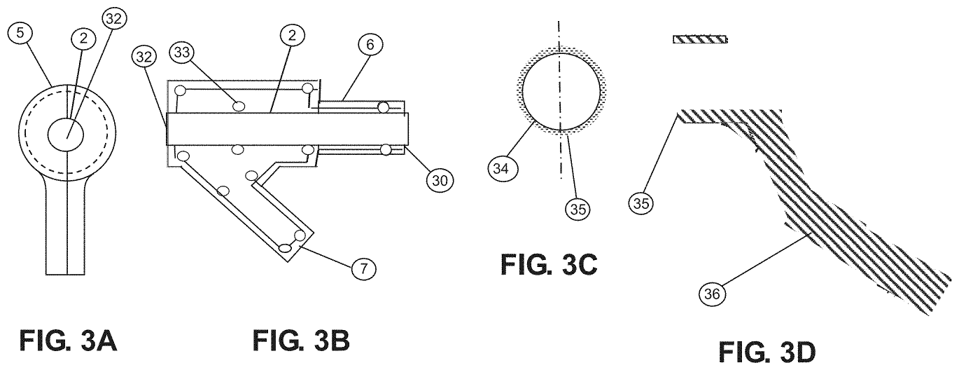

FIGS. 3(a)-3(d) illustrate a housing provided on the proximal end of the tube 2 of the endoscope according to an exemplary embodiment of the present disclosure. As shown in FIG. 3(a), a front view of the housing 5, a proximal end of the tube 2 having opening 32 is placed within the housing 5. As shown in FIG. 3(b), a cross-sectional side view of the housing 5, the housing 5 can have a clam shell 30 that can be a two-piece assembly incorporating a clam shell construction. The two halves of the claim shell 30 are cemented together along the edges, and male and female inserts 33 provided on each half of the claim shell 30 allow the two halves of the clam shell 30 to be set in place. There can be ten locking inserts 33 provided throughout the claim shell 30. During construction of the telescope, the lens tube 2 passes between the two halves of the clam shell 30 and can be cemented to it. The telescope lens tube 2 enters through the opening 32 and is fixtured to at the exit of the clam shell 30. The camera adapter 6 can be provided on the tube 2 as part of the clam shell housing.

As shown in FIG. 3(c), a rear view of the housing 5, and FIG. 3(d), a cross-sectional view of an illumination insert 36, the housing 5 contains a transmissive polymer fiber optic 36 that is wrapped around the lens tube 2 prior to its retention in the housing 5. The transmissive polymer fiber optic 36 forms an illumination ring around the lens tube 2 shown between inner diameter 34 of the polymer fiber optic 35. The illumination is supplied by an external light source connected to a light ferrule 7 that passes through the polymer fiber optic 36 and transmits light from a proximal end of the tube 2 to the distal end of the tube 2.

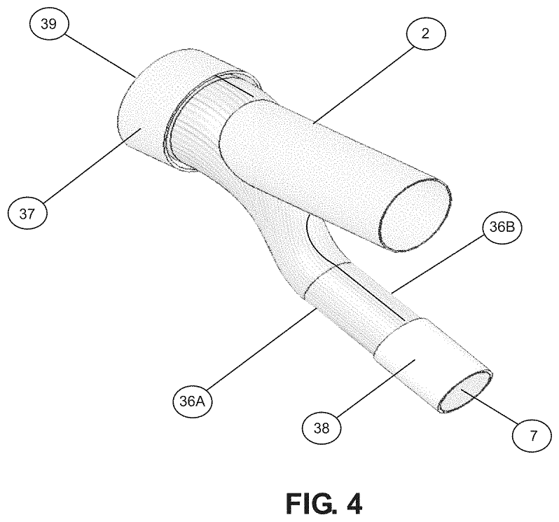

FIG. 4 illustrates an internal design of the housing 5 and of the endoscope according to an exemplary embodiment of the present disclosure. The polymer fiber optic 36 can be made from two matching high index polymer elements 36A and 36B. This assembly can create a light ring around the lens tube 2 at the proximal end of the endoscope and interfaces with an external light source terminating at the ferrule 7. Two retainers, a front retainer 39 and a rear retainer 38, can hold the two illumination elements 36A and 36B together. A focal image can be generated at the proximal end of the lens tube 2. The housing 5 provides an interface at front retainer 39 with a light lumen that extends from the housing 5 to the distal end of the endoscope.

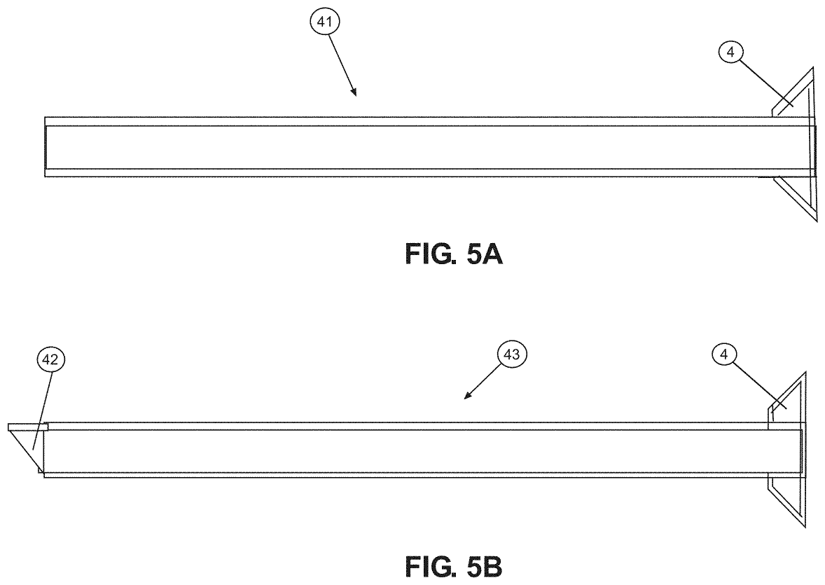

FIG. 5(a) illustrates a zero degree viewing angle embodiment of the endoscope according to an exemplary embodiment of the present disclosure, and FIG. 5(b) illustrates a thirty degree viewing angle embodiment of the endoscope according to an exemplary embodiment of the present disclosure. The zero degree viewing angle endoscope 41 can have a construction as shown in FIGS. 1-4 with a cemented flange 4 at a proximal end of the endoscope 41 connecting it to housing 5. The thirty degree viewing angle endoscope 41 can have a similar internal construction as shown in FIGS. 1-4, and has a prism 42 mounted to the distal end to create the thirty degree lumen 43. The prism allows for the thirty degree viewing angle. The illumination lumen 3 is held in place by flange 4 in addition to the frictional forces of the lumen 3 and lens tube 2.

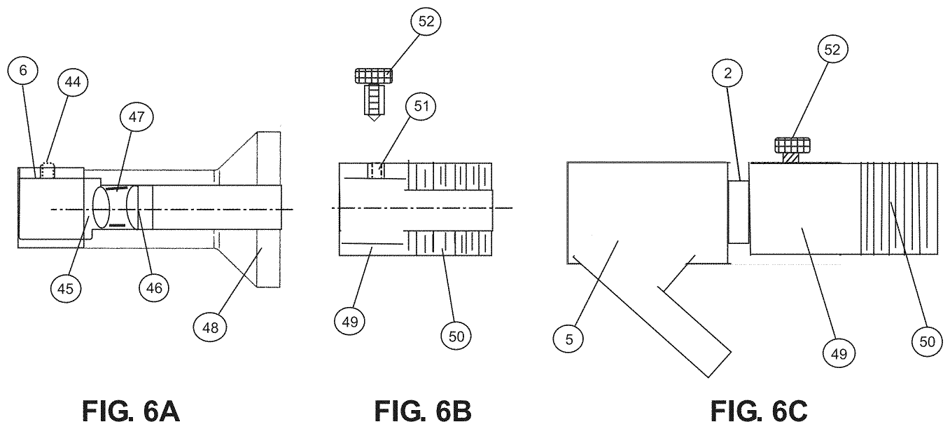

FIGS. 6(a)-6(c) illustrate an eyepiece and camera interface to be connected to the housing of the endoscope according to an exemplary embodiment of the present disclosure. As shown in FIG. 6(a), the camera adapter 6 is provided within the eyepiece assembly 48. The eyepiece assembly 48 includes a lens 45 through which the image is provided from the distal end of the endoscope, and a lens 46 to which the image is provided. The lens 45 is provided approximately 4 mm from the from the end of the lens tube 2 within the camera adapter 6. The lens 46 is separated from lens 45 a fixed distance through spacer 47. Focus can be adjusted by sliding the eyepiece assembly 48 until an image is focused for the human eye, which is when the lens 45 is approximately 4 mm from the from the end of the lens tube 2 within the camera adapter 6.

FIG. 6(b) shows a camera interface 49 that can be assembled or connected to the camera adapter 6. Once focus has been achieved by sliding the camera interface 49 into the camera adapter 6, and the set screw combination 44, 51 is lined up, the body of the eyepiece assembly 48 can be held at that position by a set screw combination 44, 51 to lock the camera interface 49 to the camera adapter 6 by tightening the set screw 52. The camera interface 49 can be threaded into a standard medical camera using “C” mount threads 50 that has matching threads. The camera can be focused by viewing the image on a high-resolution video monitor (not shown) and adjusted for best focus. Having achieved the desired image, set screw combination 44,51 are secured and the image is focused for video use. Fine turning of the camera image may also be achieved by adjusting the threads 50 of the camera interface with respect to the camera. As shown in FIG. 6(c), the camera interface 49 can be mounted to the housing 5 with the set screw 52 tightened. The camera interface 49 slides over the housing 5, until the desired focus is achieved.

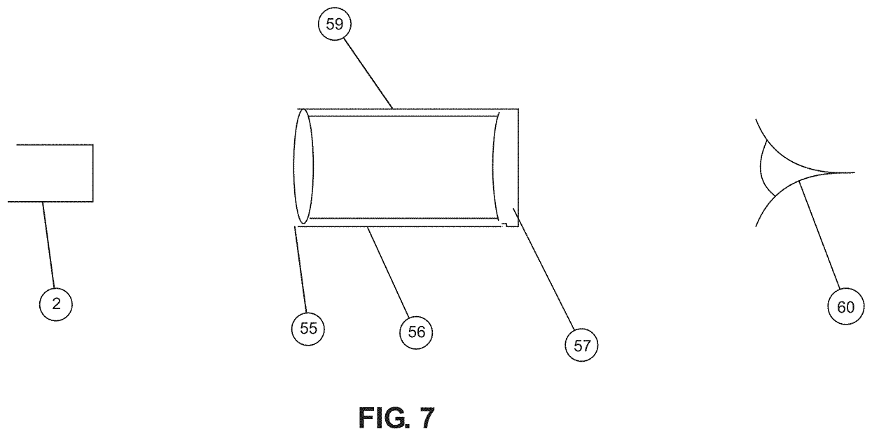

FIG. 7 illustrates an eyepiece lens assembly according to an exemplary embodiment of the present disclosure. An image exiting the tube 2 is focused approximately four millimeters from the end of the tube 2. If a camera were to be used without an eyepiece an image formed would be focused onto the photo sensitive surface of the camera and an image recorded. If an eyepiece is required, a separate optical system 56 can be provided including a singlet lens 55 spaced by a lens spacer 59 to relay an image to an eyepiece lens 57. The image on the eyepiece lens 57 can be provided approximately 12 mm from a human eye 60.

Various advantages can also be provided by the exemplary embodiments of the present disclosure. For example, in some exemplary embodiments, the disposable endoscope can be packaged into a clear pouch that can be sealed at both ends. The pouch can be preprinted with the manufacturing codes, instructions for use and manufacturer's identification in an instruction booklet. The endoscopes can be packaged in a box of ten units. Before being placed in a box for customer use, the endoscopes along with the packaging can be ETO sterilized in lot number identification. The materials used to construct the endoscope can be selected to deform to prevent any reuse by autoclaving re sterilization, thereby insuring normal single use.

The exemplary embodiments of the present disclosure can provide for a single use disposable endoscope that can be designed with six achromat double lenses and five singlet lenses contained in a polymer tube for image acquisition and one achromat for an optional eyepiece. In some exemplary embodiments, the telescope lenses can be coated with magnesium fluoride anti reflection coating, or other anti-reflective coatings.

The exemplary embodiments of the present disclosure eliminate the requirement for an eye piece for human viewing. Conventional endoscopes are typically designed with an eyepiece that requires an auxiliary optical system such as an endo coupler, or other similar optical device, to interface with a video camera. The endo coupler focuses an image from the eyepiece onto a camera's photo sensitive surface, and the image is then displayed onto a video monitor. An endo coupler is a costly optical element that must be re sterilized after each use. In modern operating rooms, human eye viewing is seldom if ever used, as video monitors have supplanted viewing through an eyepiece. The endoscope of the present disclosure is designed to focus an image directly onto the photo surface of a video camera eliminating any auxiliary optics.

Focusing the telescope image directly onto a video camera is accomplished using a “C” mount adapter for mounting a camera onto the endoscope output. The mounting adapter also functions as a means of adjusting the camera focus. In some exemplary embodiments of the present disclosure, the endoscope incorporates an optional eyepiece. In some exemplary embodiments, a proximal end of the camera adapter directly contacts a ferrule assembly that interlocks to the housing. The distal end of the endoscope can terminate with a zero power lens or an angled prism to hermetically seal the device. The exemplary embodiments can provide for an interchangeable zero degree or thirty degree viewing angle, although other viewing angles can also be provided. In some exemplary embodiments of the present disclosure, an external light source is connected to a ferrule on the endoscope. The ferrule brings the light from an external source at the proximal end and terminates at the distal end of the tube.

The exemplary embodiments of the present disclosure can provide for a disposable endoscope through the use of a simplified optical system. Furthermore, plastic lenses can be substituted for the singlet glass lenses used with minimal change in image quality with magnesium fluoride coating. In some exemplary embodiments of the present disclosure, polyether ether ketone (PEEK) can be used as the material of the lens tube in place of stainless steel. PEEK provides for high strength, chemical resistance and heat tolerance. It can easily be pigmented to be light absorbing to reduce stray light within the telescope. PEEK can also be used for the lens spacers, which can add strength to the telescope's structural integrity. In some exemplary embodiments of the present disclosure, the housing 5 is also constructed of PEEK material designed in a clam shell configuration. The two halves of the clam shell encompass the ferrule system and provide a camera mount with a means to adjust the focus. The proximal assembly clam shell is cemented together and to the lens tube to complete the telescope assembly.

The ferrule light element can be constructed of a cyclic polyolefin, an optical grade plastic that achieves a 93% optical transmission. This grade of plastic material is used because the plastic cannot survive the autoclave process and distorts in the sterilization process, thus, preventing reuse. The exemplary embodiments of the present disclosure can use plastic cylinder lumens in place of the fiber optics for illumination. In some exemplary embodiments of the present disclosure, the endoscope uses a cylinder of high index, high transmission polymer, cyclic polyolefin as the high index core material and air as the low refractive index cladding, providing the same total internal reflection phenomena as with glass fiber optics. However, the glass fibers have a higher numerical aperture (NA) and are more efficient than the plastic material that is lower in transmission. To compensate for the lower NA, the embodiment of the plastic disposable endoscope can provide for increasing the light output of the light source and diffusing the distal end of the lumen to expand the area of illumination.

The exemplary embodiments of the present disclosure provide for an interchangeable zero degree or angled (30 degree) endoscope. The lumen in the angled endoscope can be rotated continuously 360 degrees, unlike conventional fixed angled laparoscopes that have limited rotational capability. The plastic lumen has the advantage of being an extruded cylinder which permit greater flexibility to expand the applications of the laparoscope's capabilities by providing additional working channels.

A self-sealing diaphragm located at a proximal end of the working channel lumen provides a means office oriented therapy. The working channels allow therapeutic surgery using the endoscope. The working channels enable a laser ablation fiber or surgical tool to be delivered through the lumen in a direct pathway into the body eliminating the need for an additional entry port into the body. A diaphragm integrated into the proximal end of the lumen prevents back flow of particulate or fumes from exiting during surgery. The lumen can also be used to evacuate smoke, fumes and particulate post-surgery. By combining imaging and therapeutic functions into one instrument, the disposable lumen of the present disclosure addresses the need for an instrument which provides an economic imaging means for performing endoscopic surgery and provides the surgeon with access to new capabilities that are not provided by conventional laparoscopes.

Various other considerations can also be addressed in the exemplary applications described according to the exemplary embodiments of the present disclosure. For example, various materials may be used to construct the elements described in the figures. Various sizes of the endoscope can also be provided for various uses. The exemplary embodiments of the endoscope use a light transmitting polymer lumen in place of the fiber optics normally used in such devices. The extruded lumen drastically reduces cost providing for the viability of a disposable endoscope. The exemplary embodiments of the endoscope use a single layer of magnesium fluoride evaporated onto the lenses resulting in an efficient cost effective optical design. The single layer coating can achieve a level of brightness as other laparoscopes with more complicated optical systems. Coating failures are not a concern as the endoscope is a single use device and there is no need for autoclaving. The plastic lenses and single layer coating can provide for lower production costs to achieve a disposable endoscope.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure.

What is claimed is:

1. An endoscope comprising:
a tube having a proximal end and a distal end;
a lumen having a proximal end and a distal end, the lumen surrounding an outer circumference of the tube;
a first achromatic double lens provided at the distal end of the tube;
a second achromatic double lens provided within the tube and a first singlet lens provided between the first achromatic double lens and the second achromatic double lens;
a third achromatic double lens provided within the tube and a second singlet lens provided between the second achromatic double lens and the third achromatic double lens;
a fourth achromatic double lens provided within the tube and a third singlet lens provided between the third achromatic double lens and the fourth achromatic double lens;
a fifth achromatic double lens provided within the tube and a fourth singlet lens provided between the fourth achromatic double lens and the fifth achromatic double lens;
a sixth achromatic double lens provided within the tube and adjacent the fifth achromatic double lens; and
a seventh achromatic double lens provided within the tube at the proximal end of the tube and adjacent the sixth achromatic double lens.

2. The endoscope of claim 1, wherein the lumen and tube are constructed of a rigid material.

3. The endoscope of claim 1, wherein each of the achromatic double lenses and singlet lenses are made of high and low refractive index glasses having a magnesium fluoride coating.

4. The endoscope of claim 1, further comprising:
a field stop provided within the tube between the first singlet lens and the second achromatic double lens.

5. The endoscope of claim 4, further comprising:
a first spacer provided between the first achromatic double lens and the first singlet lens;
a second spacer provided between the first singlet lens and the field stop; and
a third spacer provided between the field stop and the second achromatic double lens.

6. The endoscope of claim 5, further comprising:
a fourth spacer provided between the second achromatic double lens and the second singlet lens; and
a fifth spacer provided between the second singlet lens and the third achromatic double lens.

7. The endoscope of claim 6, further comprising:
a sixth spacer provided between the third achromatic double lens and the third singlet lens; and
a seventh spacer provided between the third singlet lens and the fourth achromatic double lens.

8. The endoscope of claim 7, further comprising:
an eighth spacer provided between the fourth achromatic double lens and the fourth singlet lens; and
a ninth spacer provided between the fourth singlet lens and the fifth achromatic double lens.

9. The endoscope of claim 8, further comprising:
a tenth spacer provided between the fifth achromatic double lens and the sixth achromatic double lens; and
an eleventh spacer provided between the sixth achromatic double lens and the seventh achromatic double lens.

10. The endoscope of claim 1, further comprising:
a prism provided at the distal end of the lumen configured to provide a thirty degree viewing angle.

11. The endoscope of claim 1, further comprising:
a housing provided at the proximal end of the tube, the housing having a proximal end and a distal end; and
a cemented flange at the proximal end of the tube, the cemented flange connecting the lumen to the proximal end of the housing, wherein the tube extends within the housing.

12. The endoscope of claim 11, further comprising:
an illumination ferrule provided on the housing for providing a light pathway from the proximal end of the lumen to the distal end of the lumen.

13. The endoscope of claim 12, wherein the lumen is constructed of a high index, high transmission clear polymer to transfer light from the proximal end of the housing to the distal end of the lumen.

14. The endoscope of claim 12, wherein the housing comprises a two-piece assembly incorporating a clam shell construction having male and female inserts that lock the two pieces to each other, wherein the tube passes through the two-piece assembly.

15. The endoscope of claim 12, further comprising:
a transmissive polymer fiber optic provided in the housing around the tube, wherein illumination is provided from the illumination ferrule through the transmissive polymer fiber optic to transmit light from a proximal end of the lumen to the distal end of the lumen.

16. The endoscope of claim 15, wherein the polymer fiber optic comprises two high index polymer elements that provides a light ring around the tube.

17. The endoscope of claim 16, wherein the housing comprises:
a front retainer at a proximal end of the housing; and
a rear retainer at a distal end of the housing;
wherein the front and rear retainers hold the two high index polymer elements together.

18. The endoscope of claim 11, further comprising:
a camera adapter provided at a distal end of the housing, wherein the tube extends within the camera adapter;
an eyepiece assembly connected to the camera adapter; and
a camera interface provided within the eyepiece assembly and secured to the eyepiece assembly.

19. The endoscope of claim 18, further comprising:
a first lens provided within the eyepiece assembly adjacent the camera adapter;
a spacer provided adjacent the first lens; and
a second lens provided adjacent the spacer, wherein an image is provided from the seventh achromatic double lens through the first lens of the eyepiece assembly to the second lens of the eyepiece assembly.

20. The endoscope of claim 19, further comprising:
threads within the camera interface for securing a camera to the camera interface to receive the image from the second lens of the eyepiece assembly.
